(19) United States
(12) Patent Application Publication
(10) Pub. No.: US 2026/0062523 A1
(43) Pub. Date: Mar. 5, 2026

(54) FORMULA DESIGN AND OPTIMIZATION METHOD FOR CROSSLINKED POLYETHYLENE INSULATING MATERIAL OF HIGH-VOLTAGE ALTERNATING-CURRENT CABLE

(71) Applicant: ELECTRIC POWER RESEARCH INSTITUTE. CHINA SOUTHERN POWER GRID, Guangzhou (CN)

(72) Inventors: Shuai HOU, Guangzhou (CN); Mingli FU, Guangzhou (CN); Lei JIA, Guangzhou (CN); Lingmeng FAN, Guangzhou (CN); Wenbo ZHU, Guangzhou (CN); Baojun HUI, Guangzhou (CN); Yunpeng ZHAN, Guangzhou (CN); Bin FENG, Guangzhou (CN); Jie LIU, Guangzhou (CN); Chunyang LI, Guangzhou (CN)

(73) Assignee: ELECTRIC POWER RESEARCH INSTITUTE. CHINA SOUTHERN POWER GRID, Guangzhou (CN)

(21) Appl. No.: 19/107,147

(22) PCT Filed: Nov. 9, 2022
(86) PCT No.: PCT/CN2022/130845

§ 371 Date: Feb. 27, 2025

(30) Foreign Application Priority Data
Sep. 2, 2022 (CN) 202211070878.3

(51) Int. Cl. C08J 3/24; C08F 8/00; G01N 33/44; G01R 31/12

(52) U.S. Cl. CPC C08J 3/24; C08F 8/00; G01N 33/442; G01R 31/1263; C08F 2810/20; C08J 2323/06

(57) ABSTRACT

A method includes: step 1: determining initial elements of the formulation, including types of a base resin, an antioxidant, and a crosslinking agent; step 2: testing a sample prepared by hot press molding the pure base resin, and obtaining an optimized base resin sequentially through qualitative evaluation indicator I and quantitative evaluation indicator I; step 3: preparing multiple groups of crosslinking agent/antioxidant/base resin blends, and obtaining an optimized crosslinking agent formulation sequentially through qualitative evaluation indicator II and quantitative evaluation indicator II; step 4: preparing multiple groups of crosslinking agent/antioxidant/base resin blends, and obtaining an optimized antioxidant formulation sequentially through qualitative evaluation indicator III and quantitative evaluation indicator III; and step 5: verifying through electrical properties, if parameter measurement results all meet requirements, the formulation of the material is considered as an optimized formulation, and if not, returning to test a next candidate to be tested.

### CROSS-REFERENCE TO RELATED APPLICATIONS

This application is an U.S. national phase application under 35 U.S.C. § 371 based upon international patent application No. PCT/CN2022/130845 filed on Nov. 9, 2022, which itself claims priority to Chinese patent application No. 202211070878.3, filed on Sep. 2, 2022, entitled “FORMULA DESIGN AND OPTIMIZATION METHOD FOR CROSSLINKED POLYETHYLENE INSULATING MATERIAL OF HIGH-VOLTAGE ALTERNATING-CURRENT CABLE”. The contents of the above identified applications are hereby incorporated herein in their entireties by reference.

### TECHNICAL FIELD

The present disclosure relates to the technical field of power transmission, and in particular, to a formulation design and optimization method for crosslinked polyethylene insulating material of high-voltage alternating-current cable.

### BACKGROUND

For urban power supply, the use of high-voltage overhead lines is not advisable. Crosslinked polyethylene (XLPE) insulated high-voltage cables have advantages such as a small footprint, high safety, and resistance to weather impacts. Therefore, XLPE insulated high-voltage alternating current cables have become one of the most important routes for power transmission. The formulation of XLPE insulating materials for high voltage mainly includes a low-density polyethylene (LDPE) base resin, a crosslinking agent, and an antioxidant.

For XLPE insulating materials for a voltage of 35 kV and above, to ensure excellent insulating performance, dicumyl peroxide (DCP) is typically used as the crosslinking agent, which initiates and completes the crosslinking reaction under heat. The content of the crosslinking agent DCP significantly affects the performance of the XLPE insulating material for high-voltage cables when keeping the base resin unchanged. If the content of the crosslinking agent is too low, the material may not meet the production efficiency for crosslinking, potentially leading to insufficient crosslinking of material, which negatively impacts the mechanical properties and long-term thermal resistance of the material. To meet the performance indicators internationally commonly used for XLPE insulating cable materials, the amount of peroxide added in the crosslinking process of the XLPE insulating cable material in China is 1 time to 1.5 times higher than that in foreign countries, with the amount of DCP in domestic 35 kV XLPE insulating materials sometimes reaching as high as 2 to 2.2 phr. However, an excessive amount of DCP can also lead to a series of problems: first, it can cause over-crosslinking of the material, resulting in decreased mechanical properties; second, the amounts of by-products of crosslinking, such as acetophenone and cumyl alcohol, are increased, which imposes stricter requirements on the degassing process of the insulating cable layer, significantly affecting the actual production efficiency of cables and increasing production costs; third, some by-products of crosslinking cannot be removed or exhausted through the degassing process, leading to the formation of micro-pores in the cable insulating material, which significantly reduces the overall performance of the cable insulating layer. Therefore, reducing the amount of DCP added is an important way to improve the performance of XLPE insulating cable materials while ensuring their excellent properties.

As the main insulating material for cables, XLPE inevitably operates in high-temperature environments during production and application, making it susceptible to aging under the influence of heat and oxygen, which affects its electrical properties, mechanical properties, and thermal stability, thereby reducing the lifespan of cables. Antioxidants, as commonly used additives in polymer materials, can delay the oxidative aging of polymers and are essential components for XLPE insulating cable materials. Different antioxidants and their contents have varying effects on the electrical, thermal, and mechanical properties of XLPE insulating cable materials, and their resistances to thermal oxidative aging also differs significantly. Additionally, the primary mechanism of antioxidants is to scavenge active free radicals in the polymer, while the thermal crosslinking reaction of XLPE can only be carried out under the action of highly active free radicals generated by DCP, thereby the antioxidants can significantly influence the crosslinking reaction behavior of the material.

The formulation design of high-voltage crosslinked polyethylene insulating materials mainly includes three major parts: determining an LDPE base resin, designing an anti-aging formulation, and designing a crosslinking formulation. In the past, in order to design a formulation for high-voltage crosslinked polyethylene insulating materials, comprehensive experiments were typically conducted on multiple compositions of various LDPE resins, various antioxidants, and various crosslinking agents, followed by a thorough comparison and analysis of the performances of the materials with different formulations to obtain a qualified formulation. As mentioned above, the performances of the crosslinked material and the performances of the LDPE base resin before crosslinking are significantly different, and the two important formulation additives, crosslinking agents and antioxidants, inherently influence each other, apparently making the formulation design of high-voltage crosslinked polyethylene insulating materials to be a complex problem. Theoretically, the orthogonal test method can be employed to solve the complex problem. The orthogonal test method in principle has the advantages such as fewer experimental runs, good results, simple process, convenience in use, and high efficiency. However, the performance evaluation on the insulating materials involves multiple aspects, including crosslinking performance, mechanical properties, electrical properties, aging performance, and so on. The mechanisms of interaction among crosslinking agents, antioxidants, and macromolecules are complex, making the orthogonal test method imprecise and unable to determine experimental laws or yield reliable results.

Currently, there are two main forms for determining formulation of materials: one is based on national standards, through a large number of non-systematic and unstructured experimental explorations to obtain preliminary formulations; and the other is based on commonly used formulations of the XLPE insulating materials without optimizing the formulation design according to the specific characteristics of the used material. The former method suffers from obvious blindness due to the lack of systematic design, resulting in significant waste of time, manpower, and material resources. In the latter method, since the designations and manufacturers of the specific base resins, antioxidants, and crosslinking agents adopted are different, which lead to significant differences in material performances, optimal material formulations cannot be obtained when other commercial materials are adopted without considering the influence of differences in raw materials.

In summary, the formulation design and optimization method for crosslinked polyethylene insulating materials is key to improve material performances and upgrade products. However, there is a lack of systematic formulation design and optimization methods currently. Conventional techniques often involve blind and large-scale experiments, which has a strong inhibitory effect on shortening the research and development cycle of insulation materials and improving the performance of insulation materials.

### SUMMARY

According to various embodiments of the present disclosure, a method for designing and optimizing a formulation of an XLPE insulating material for a high-voltage alternating current cable is provided.

A method for designing and optimizing a formulation of an XLPE insulating material for a high-voltage alternating current cable, includes the following steps:
step 1: determining initial elements of the formulation, including types of a base resin, an antioxidant, and a crosslinking agent;
step 2: based on the base resin selected in the step 1, testing a sample prepared by hot press molding the pure base resin, and conducting optimization sequentially through two levels of indicators: qualitative evaluation indicator I and quantitative evaluation indicator I, to obtain an optimized base resin, wherein the qualitative evaluation indicator I includes a rheological property parameter, a tensile strength parameter, an elongation at break parameter, and dielectric loss angle tangent and relative dielectric constant parameters, and the quantitative evaluation indicator I comprises a two-parameter Weibull distribution parameter of alternating current breakdown field strength;
step 3: preparing multiple groups of crosslinking agent/antioxidant/base resin blends, wherein mass fractions of the antioxidant and the base resin in the multiple groups remain the same, while mass fractions of the crosslinking agent in the multiple groups are arranged in an ascending sequence, testing samples of the blends from each group, and conducting optimization through two levels of indicators: qualitative evaluation indicator II and quantitative evaluation indicator II, to obtain an optimized crosslinking agent formulation, wherein the qualitative evaluation indicator II includes a gel content, a thermal elongation, a tensile strength parameter, and an elongation at break parameter, and the quantitative evaluation indicator II includes a crosslinking gas production property parameter;
step 4: preparing multiple groups of crosslinking agent/antioxidant/base resin blends, wherein mass fractions of the crosslinking agent and the base resin in the multiple groups remain the same, while mass fractions of the antioxidant in the multiple groups are arranged in an ascending sequence, testing samples of the blends from each group, and conducting optimization sequentially through two levels of indicators: qualitative evaluation indicator III and quantitative evaluation indicator III, to obtain an optimized antioxidant formulation, wherein the qualitative evaluation indicator III includes the gel content, the thermal elongation, and the tensile strength parameter and the elongation at break parameter before and after aging, and the quantitative evaluation indicator III includes a characteristic parameter of a crosslinking reaction kinetic curve; and
step 5: verifying through electrical properties, wherein if parameter measurement results all meet requirements, the formulation of the material is considered as an optimized formulation; if not, returning to test a next candidate to be tested.

In the step 2, 3, or 4, the qualitative evaluation involves judging whether the parameter measurement results meet the qualitative evaluation indicator I, the qualitative evaluation indicator II, or the qualitative evaluation indicator III, if yes, the material is considered qualified, and if not, that material is abandoned, and a next candidate material is subjected to the qualitative evaluation, until all candidate materials have undergone testing for the qualitative evaluation indicator I, the qualitative evaluation indicator II, or the qualitative evaluation indicator III, and the material meeting the qualitative evaluation indicator is selected to undergo testing for the quantitative evaluation indicator I, the quantitative evaluation indicator II, or the quantitative evaluation indicator III.

In an embodiment, the qualitative evaluation indicator I includes: the rheological property parameter measured with a rotational rheometer, the tensile strength parameter and the elongation at break parameter measured based on a stress-strain test, and the dielectric loss angle tangent and relative dielectric constant parameters measured based on high-voltage Schering bridge.

In an embodiment, the quantitative evaluation indicator I includes the two-parameter Weibull distribution parameter of the alternating current breakdown field strength measured with a cylindrical electrode.

With a characteristic breakdown strength as a first parameter a and a shape parameter as a second parameter b, priority ranking on different resins is conducted according to values of a; the greater the a, the higher the priority; and with a the same, b is used to further refine the priority ranking, and for materials with the same a, the smaller the b, the higher the priority.

After the priority ranking for all resins for optimization is completed, the resin ranked first proceeds to the step 3, while the resins ranked later serves as candidates to be tested.

In an embodiment, in the step 3, samples prepared from x phr crosslinking agent/0.3 phr antioxidant 300/base resin blends are tested and subjected to the optimization through two levels of indicators: the qualitative evaluation indicator II and the quantitative evaluation indicator II;
wherein x is a custom variable sequence: x₁, x₂, x₃, x₄ . . . xₙ₁, indicating the mass fraction of the crosslinking agent, x≥0, and n₁ denotes a length of the sequence, i.e., a number of groups of the blends.

In an embodiment, the qualitative evaluation indicator II includes: gel content and thermal elongation parameters characterizing the degree of crosslinking; and the tensile strength parameter and the elongation at break parameter obtained based on the stress-strain test.

In an embodiment, the quantitative evaluation indicator II includes a crosslinking gas production property parameter. Priority ranking is conducted on materials of all components. The better the gas production property, the smaller the degassing residual amount, the higher the priority. When the degassing residual amounts are the same, the smaller the x, the higher the priority. After completing the priority ranking for all materials for optimization: x₁, x₂, x₃, x₄ . . . xₙ₁, where n₁ denotes a length of the sequence, the material ranked first proceeds to the step 4, while the materials ranked later serve as candidates to be tested.

In an embodiment, in the step 4, based on mechanism of action and synergistic effects, the antioxidant is used alone, or two types of antioxidants are used in combination.

In an embodiment, the qualitative evaluation indicator III includes: gel content and thermal elongation parameters characterizing the degree of crosslinking; and the tensile strength parameter and the elongation at break parameter before and after aging obtained based on an air aging property test.

In an embodiment, the quantitative evaluation indicator III includes: the characteristic parameter of the crosslinking reaction kinetic curve. Priority ranking is conducted on materials of all components. The further to the right the peak point of the crosslinking reaction kinetic curve, the higher the priority. When the crosslinking reaction kinetic curves coincide, the higher the gel content in the qualitative evaluation indicator III, the higher the priority. When the crosslinking reaction kinetic curves coincide and the gel contents in the qualitative evaluation indicator III are the same, the higher the elongation at break in the qualitative evaluation indicator III, the higher the priority. After the priority ranking for all materials is completed, the material ranked first proceeds to the step 5.

Details of one or more embodiments of the present disclosure are presented in the drawings and description below. Further features, objectives, and advantages of the present disclosure will become apparent from the description, drawings and claims.

### BRIEF DESCRIPTION OF THE DRAWINGS

In order to more clearly illustrate technical solutions in the embodiments of the present disclosure or conventional technologies, the accompanying drawings required for use in the description of the embodiments or the conventional technologies are briefly introduced below. Obviously, the accompanying drawings described below are only some embodiments of the present disclosure, and for those of ordinary skill in the art, other drawings can be derived from these drawings without creative efforts.

FIG. 1 is a flowchart of a method for designing and optimizing a formulation of a crosslinked polyethylene insulating material for a high-voltage alternating current cable in some embodiments.

### DETAILED DESCRIPTION

The technical solutions in embodiments of the present disclosure are clearly and completely described in the following with reference to the accompanying drawings in the embodiments of the present disclosure. Apparently, the described embodiments are merely some rather than all of the embodiments of the present disclosure. All other embodiments obtained by a person of ordinary skill in the art based on the embodiments of the present disclosure without creative efforts shall fall within the protection scope of the present disclosure.

To address the problems existing in the conventional art, the present disclosure aims to provide a method for designing and optimizing a formulation of a crosslinked polyethylene insulating material for a high-voltage alternating current cable. The method is intended to address the following problems existing in the design and optimization of the formulation of the crosslinked polyethylene insulating material for the high-voltage alternating current cable in the prior art: (1) lack of targeting and systematicity; (2) long experimental period and large experimental workload; (3) formulation design only referring to related basic standards, and lack of evaluation and optimization on key properties.

The present disclosure will be further described below in conjunction with the accompany drawings. The following embodiments are intended only to more clearly illustrate the technical solutions of the present disclosure but not to limit the scope of protection of the present disclosure.

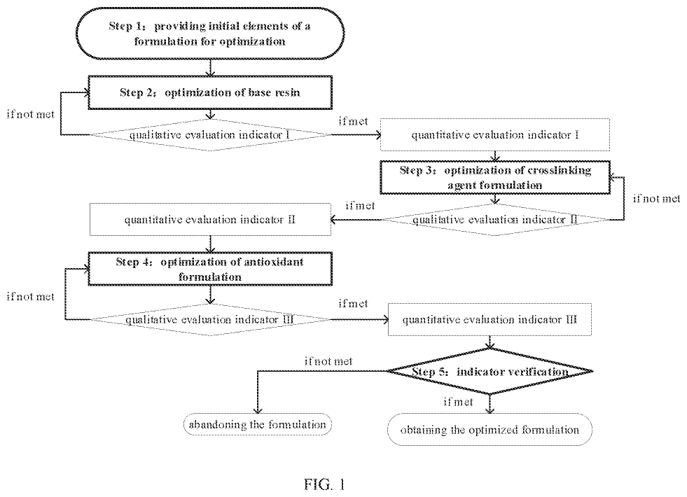

As shown in FIG. 1, a first embodiment of the present disclosure provides a method for designing and optimizing a formulation of an XLPE insulating material for a high-voltage alternating current cable, including the following steps.
Step 1: determining initial elements of the formulation, including types of a base resin, an antioxidant, and a crosslinking agent.

Specifically, designations of the base resins for optimization are determined, designations of the antioxidants are determined, and designation of the crosslinking agent DCP is determined, with the resins respectively denoted as L₁, L₂, L₃ . . . , the antioxidants respectively denoted as AO₁, AO₂, AO₃ . . . ; and the crosslinking agents respectively denoted as D₁, D₂, D₃ . . . .
Step 2: optimization of the base resin. Based on the base resins selected in the step 1, samples prepared by hot press molding the pure base resins are tested, and the optimization is conducted sequentially through two levels of indicators: qualitative evaluation indicator I and quantitative evaluation indicator I.

The qualitative evaluation involves judging whether the parameter measurement results meet the qualitative evaluation indicator I. If yes, the material is considered qualified; if not, that material is abandoned, and a next candidate resin material is subjected to the qualitative evaluation, until all candidate resin materials have undergone testing for the qualitative evaluation indicator I. The resin material that meets the qualitative evaluation indicator I is then selected to test the quantitative evaluation indicator I.

In an embodiment of the present disclosure, the qualitative evaluation indicator I includes: a rheological property parameter measured with a rotational rheometer; a tensile strength parameter and an elongation at break parameter measured based on a stress-strain test; and dielectric loss angle tangent and relative dielectric constant parameters measured based on high-voltage Schering bridge. Meeting all of the above indicators is considered qualified, allowing proceeding to the quantitative evaluation indicator I.

It should be noted that the aforementioned qualitative evaluation indicator I is described as an embodiment of the technical solutions of the present disclosure. Those skilled in the art may set more stringent qualitative evaluation indicator I based on design requirements, for example, but not limited to, adding more judgment conditions for the qualitative evaluation indicator I, or raising the qualification standard for each specific indicator. Similarly, it should be understood that in the subsequent technical solutions of the present disclosure, all qualitative and quantitative evaluation indicators can be adaptively adjusted by those skilled in the art.

The specific requirements for the qualitative evaluation indicator I are set out below:

| | | | Dielectric property |
|---|---|---|---|
| | | Parameter based | parameter at |
| | | on stress-strain | working frequency |
| Rheological property parameter | | test | (50 Hz, 20° C.) |
| shear frequency | complex viscosity Pa · s | tensile | relative dielectric |
| (0.01 rad/s, 120° C.) | (1.3 ± 0.8) × 10⁵ | strength ≥12 | constant ≤2.35 |
| (0.01 rad/s, 150° C.) | (4.7 ± 0.7) × 10⁴ | MPa | |
| (0.01 rad/s, 180° C.) | (1.75 ± 0.5) × 10⁴ | | |
| (1 rad/s, 120° C.) | (2.3 ± 0.5) × 10⁴ | | |
| (10 rad/s, 120° C.) | (6.1 ± 0.5) × 10³ | | |

The quantitative evaluation indicator I includes a two-parameter Weibull distribution parameter of the alternating current breakdown field strength measured with a cylindrical electrode. Different resins for optimization are respectively tested, using the characteristic breakdown strength (a scale parameter) as a first parameter a and the shape parameter as a second parameter b. Priority ranking is conducted on the different resins according to the values of a. The greater the a, the higher the priority. With the same a, b is used to further refine the priority ranking. For materials with the same a, the smaller the b, the higher the priority. After completing the priority ranking for all resins for optimization, the resin ranked first proceeds to the step 3, while the resins ranked later serve as candidates to be tested.

It should be understood that in engineering practice, one or more types of the base resins, the antioxidants, and the crosslinking agents can be selected in the step 1, which does not affect the implementation of subsequent steps. For example, but not limited to, if only one type of the base resin is selected in the step 1, the quantitative evaluation ranking process in the step 2 can be acquiescently skipped, directly proceeding to the subsequent steps.

It should be noted that the existing specific indicators for high-voltage crosslinked polyethylene insulating materials are primarily based on general standards in the industry, which only addresses a limited number of property criteria. Key properties such as the rheological property, the gas production property, and the crosslinking kinetic curve, which directly depend on the formulation of the material, are not specified in the standards. Among these, the rheological property determines the extrusion processibility of the material and the uniformity of the insulating structure; the gas production property determines the time required for degassing after crosslinking of the material and the effectiveness of the degassing process, ultimately affecting the long-term operational stability of the material; and the crosslinking kinetic curve determines the resistance to scorching of the material and limits the maximum extrusion length of the cable insulation material.

Furthermore, existing general standards in the industry only specify requirements for properties of materials after crosslinking, without providing specific requirements for the base resin. Further, there is currently a lack of guiding methods. This leads to blindness in the initial design of formulation of the materials, as the impurity content and gel point content of the base resin play a decisive role in the electrical properties of the crosslinked polyethylene insulating materials. One of the improvements of the present disclosure over the conventional art is that the present disclosure provides requirements for these key properties of the base resin, which avoids failures in formulation due to insufficient key property assessments, and significantly increases the success rate of formulation design.

More specifically, one of the prominent substantive features of the present disclosure and the outstanding beneficial effects resulted are as follows. In the qualitative evaluation indicator I of the present disclosure, the rheological property measured based on the rotational rheometer is used in the present disclosure to qualitatively evaluate the base resins, which is more specific than conventional base resin performance indicators (such as density and melt index) and fully considers the requirements of the cable insulating material manufacturing process and the requirements for the base resin in the cable insulating layer manufacturing process. The viscosity at low shear frequency is used to limit the zero-shear viscosity of the material to prevent eccentricity after extrusion due to insufficient zero-shear viscosity of the material. The viscosity at high shear frequency is used to limit the extrusion processibility of the material to prevent problems of excessive or insufficient extrusion volume and poor extrusion quality. The limitation on tensile strength and elongation at break is to prevent the problem that the tensile strength and elongation at break cannot meet the requirements after cross-linking of the material, and to further limit the macromolecular structure of the base resin.

In the qualitative indicator I of the present disclosure, the Weibull distribution parameter of the breakdown field strength imposes further requirements on the purity of the material to prevent the presence of large-sized impurities or gel points. Among various resins to be selected, the resin with higher purity is preferentially selected based on electrical properties. Additionally, the dielectric loss angle tangent further limits the molecular structure purity of the material to prevent the presence of a large number of polar groups in the macromolecules, to ensure that the base resin has sufficient margin in the dielectric loss angle tangent, and to avoid the problems that the dielectric loss angle tangent exceeds the requirements after the subsequent addition of polar molecules such as antioxidant and crosslinking agent to the base resin.
Step 3: optimization of the crosslinking agent formulation. Multiple groups of crosslinking agent/antioxidant/base resin blends are prepared, wherein the mass fractions of the antioxidant and the base resin in the multiple groups remain the same, while the mass fractions of the crosslinking agent in the multiple groups are arranged in an ascending sequence. Samples of the blends from each group are tested and optimization is conducted sequentially through two levels of indicators: qualitative evaluation indicator II and quantitative evaluation indicator II.

In an embodiment of the present disclosure, samples prepared from x phr crosslinking agent/0.3 phr antioxidant 300/base resin blends are tested in two steps and are optimized through two levels of indicators: qualitative evaluation indicator II and quantitative evaluation indicator II. x is a custom variable sequence: x₁, x₂, x₃, x₄ . . . xₙ₁, indicating the mass fraction of the crosslinking agent, x≥0, n₁ denotes the length of the sequence, i.e., the number of groups of the blends. In some examples, the individual values of x form an arithmetic progression, with the minimum common difference not less than 0.01 phr and not greater than 0.2 phr.

It should be noted that due to the wide variety of antioxidants, if a technical solution combining a primary antioxidant with a secondary antioxidant is adopted, the selection of formulations becomes more complex. As one of the improvements of the present disclosure over the conventional art, the antioxidant 300 is selected with the beneficial technical effects that can be achieved at least in that, the selection of the antioxidant influences both the optimization of DCP content and subsequent processes. Therefore, when optimizing the DCP content, the cooperative antioxidant formulation should be representative and feasible, so as to ensure the reliability of the DCP content optimization results while reducing workload and simplifying the formulation optimization process.

The antioxidant 300 is a commonly used additive in XLPE insulation materials, functioning as both the primary and secondary antioxidants. Furthermore, the antioxidant formulation with 0.3 phr antioxidant 300 is a mature solution proven feasible through the engineering practice. The aim of using 0.3 phr antioxidant 300 for cooperation is to simplify the design process and yield a reliable DCP content. In contrast, if any of the antioxidant formulations in the candidate formulations is used for cooperation with the optimization of DCP content, considering that the candidate formulations may not be representative and even fail to meet usage conditions, the obtained results may have no universality and cause significant deviations in the design of DCP contents and in further optimization design of antioxidant formulations.

In an embodiment, the qualitative evaluation indicator II includes: two parameters, i.e., gel content and thermal elongation, which characterize the degree of crosslinking; and tensile strength and elongation at break parameters obtained based on a stress-strain test. The testing method and specific parameter requirements for the qualitative evaluation indicator only need to comply with the relevant general standards for target-voltage-level crosslinked polyethylene insulating materials. Meeting all of the above indicators is considered qualified. If not, that formulation is abandoned, and the qualitative evaluation moves on to the next candidate crosslinking agent formulation, until all candidate formulations have undergone testing for the qualitative evaluation indicator II. The formulation that meets the qualitative evaluation indicator II is then selected to test the quantitative evaluation indicator II.

The quantitative evaluation indicator II includes the crosslinking gas production property parameter. A priority ranking is conducted on materials of all components. The better the gas production property, the smaller the degassing residual amount, the higher the priority. When the degassing residual amounts are the same, the smaller the value of x, the higher the priority. All materials for optimization are subjected to the priority ranking: x₁, x₂, x₃, x₄, . . . xₙ₁, where n1 denotes the length of the sequence. The material ranked first proceeds to the step 4. The materials ranked later serve as candidates to be tested.

In an embodiment, the testing method for the crosslinking gas production property in the qualitative indicator II includes: weighing approximately 5 g to 10 g of the formulation material to be tested using a precision balance, accurately measuring the weight before the reaction, conducting the hot press molding and the crosslinking reaction in a platen vulcanizer, then placing the material in a vacuum oven at −0.1 MPa and 80° C. for degassing for 72 hours, and obtaining the gas production amounts at different degassing times. The theoretical gas production amount is first calculated, defined as the percentage of DCP added relative to the mass of the blend. The gas production amount is defined as the percentage of material loss after crosslinking and degassing relative to the initial total mass of the blend. The degassing residual amount is expressed by the following formula:

$$degassing residual amount = \frac{theoretical gas production amount - gas production amount at 72 h}{theoretical gas production amount}$$

Another one of the prominent substantive features of the present disclosure and the outstanding beneficial effects resulted therefrom are as follows. In the step of optimizing the crosslinking agent formulation in the present disclosure, the qualitative evaluation indicator II is used to limit the crosslinking property and the mechanical property of the material, thereby limiting the lower limit of the amount of the crosslinking agent. Additionally, the quantitative evaluation indicator II, i.e., gas production during crosslinking is established to optimize the formulation with low gas production or high degassing efficiency, which can be seen as achieving the purpose of exploring the upper limit of the amount of crosslinking agent. Consequently, this formulation optimization step can address many problems caused by an excessive amount of crosslinking agent in the material.
Step 4: Optimization of antioxidant formulation. Multiple groups of crosslinking agent/antioxidant/base resin blends are prepared, wherein the mass fractions of the crosslinking agent and the base resin in the multiple groups remain the same, while the mass fractions of the antioxidant in the multiple groups are arranged in an ascending sequence. Samples of the blends from each group are tested, and the optimization is conducted sequentially through two levels of indicators: qualitative evaluation indicator III and quantitative evaluation indicator III.

In an embodiment, based on the mechanism of action and synergistic effects, the antioxidant can be used alone, or two types of antioxidants can be selected and used in combination. Taking two types of antioxidants used in combination as an example, samples of the y phr antioxidant AO₁/z phr antioxidant AO₂/xₙ phr crosslinking agent/base resin blends are tested in two steps, wherein xₙ is the mass fraction of the crosslinking agent obtained in the step 3, y is a custom variable sequence for the first antioxidant: y₁, y₂, y₃, . . . yₙ₂, z is a custom variable sequence for the second antioxidant: z₁, z₂, z₃, zₙ₃, both y and z represent the mass fractions of the antioxidants, both y and z are greater than or equal to 0, with the condition that they are not both zero at the same time, and n₂ and n₃ indicate the lengths of the sequences. The optimization is conducted through two levels of indicators: qualitative evaluation indicator III and quantitative evaluation indicator III. In some examples, y and z are arithmetic sequences, with the minimum common difference not less than 0.01 phr and not greater than 0.3 phr.

In an embodiment, the qualitative evaluation indicator III includes: two parameters, i.e., gel content and thermal elongation which characterize the degree of crosslinking; and tensile strength and elongation at break parameters before and after aging obtained based on an air ageing property test. Meeting all of the above indicators is considered qualified; if not, the formulation is abandoned, and the qualitative evaluation moves on to the formulation material of the next candidate antioxidant, until all candidate formulations have undergone testing for the qualitative evaluation indicator III. The formulations that meet the qualitative evaluation indicator III are selected to proceed to the quantitative evaluation indicator III.

The quantitative evaluation indicator III includes the characteristic parameter of the crosslinking reaction kinetic curve. A priority ranking is conducted on materials of all components. The further to the right the peak point of the kinetic curve of the crosslinking reaction (with time as the horizontal axis), the higher the priority. When the crosslinking reaction kinetics curves coincide, the higher the gel content in the qualitative evaluation indicator III, the higher the priority. When the crosslinking reaction kinetic curves coincide and the gel contents in the qualitative evaluation indicator III are the same, the higher the elongation at break in the qualitative evaluation indicator III, the higher the priority. After completing the priority ranking for all materials for optimization, the material ranked first proceeds to the step 5, and the materials ranked later serve as candidates to be tested.

Another prominent substantive feature of the present disclosure and the significant beneficial effects it brings are as follows. In the present disclosure, based on the dependence of these key properties on the formulations of the materials (such as antioxidant or crosslinking agent), the property testing step is conducted at the optimal location in the screening process, significantly reducing the number of repetitive tests and unnecessary duplicate testing, which not only reduces the overall workload but also improves the efficiency of formulation design and optimization. For example, the kinetic curve of the crosslinking reaction is dependent on both the antioxidant and the crosslinking agent. However, in the present disclosure, the kinetic curve of the crosslinking reaction is considered in the quantitative evaluation indicator III, but not in the quantitative evaluation indicator II, thereby reducing the workload.
Step 5: verifying through electrical properties. If parameter measurement results all meet the requirements, the formulation of the material is considered as an optimized formulation, if not, returning to test a next candidate to be tested.

In an embodiment, the verification of the electrical properties is a qualitative evaluation, specifically including: conductivity, dielectric loss factor, relative dielectric constant, and alternating current breakdown strength.

In an embodiment, when the above parameters all meet the requirements, the formulation of the material is considered as an optimized formulation. If any one of the parameters does not meet the requirements, that formulation is abandoned, and the process returns to the quantitative evaluation indicator III, continuing to verify the electrical properties based on the priority. During the verification testing, the parameter that does not meet the requirement of the previous formulation is tested first, followed by other parameters. If none of the formulations in the qualitative evaluation indicator III pass the electrical property verification, the process returns to the quantitative evaluation indicator II for further testing based on the priority. If none of the formulations in the quantitative evaluation indicator II yield a final formulation, the process returns to the quantitative evaluation indicator I. If none of the formulations in the quantitative evaluation indicator I yield a final formulation, the initial formulation elements are redefined, and the formulation design and optimization process is restarted. The requirements for the electrical property parameters in the step 5 serve as the target objectives for the present formulation design and optimization, which can be based on custom specific requirements or industry standards.

One of the prominent substantive features of the present disclosure and the significant beneficial effects it brings are as follows. The comprehensive testing of the electrical properties serves only as the final verification for individual formulations, thereby avoiding repetitive electrical property tests with a low reference value.

The beneficial effects of the present disclosure are as follows. Compared to the conventional art, as previously mentioned, the present disclosure provides specific requirements for the key properties of the base resin, provides clear guiding methods, and fully limits the impurity content, the gel point content, and the polar groups of the base resin based on the electrical properties, avoiding the blind selection for the base resin and preventing the failures of formulations due to insufficient assessment of key properties, which reduces the waste of subsequent experimental workload and significantly increases the success rate of formulation design.

Based on the inventor's long-term experimental exploration, the optimization of the crosslinking agent formulation cannot overlook the influence of the added antioxidant. In the optimization of the crosslinking agent formulation in the present disclosure, 0.3 phr of antioxidant 300 is used for cooperation. The unique molecular structure of this antioxidant allows it to function as both the primary and secondary antioxidants, demonstrating representativeness and compatibility in functionality. Therefore, the optimization phase of the crosslinking agent is more universal, providing a reliable crosslinking agent formulation optimization structural foundation for subsequent antioxidant formulation optimization, thus reducing potential waste of human resources, material resources, and time.

The three levels of quantitative evaluation indicators in the present application are used to optimize the base resin, the crosslinking agent formulation, and the anti-aging formulation, thereby promoting the formulation design towards high electrical resistance, easy degassing, scorch resistance, and aging resistance according to the weighted priority. This enhances the key properties that are of general concern in the research and development of insulating materials for high-voltage alternating current cables, facilitates the research and development of crosslinked polyethylene insulating materials for alternating current cables at higher voltage levels, and aids in the iterative upgrade of crosslinked polyethylene insulating materials for fixed-voltage grade alternating current cables.

To describe the implementation steps of the present disclosure and the beneficial technical effects that can be achieved clearer, a specific example is introduced below.

A method for designing and optimizing a formulation of an XLPE insulating material for a high-voltage alternating current cable, including the following steps.
Step 1: the initial formulation elements were determined, including: one type of base resin LDPE denoted as L₁; two types of antioxidants, the antioxidant 1010 and the antioxidant 1035, respectively denoted as AO₁ and AO₂; and a crosslinking agent DCP, denoted as D₁.
Step 2: optimization of the base resin. Since this example did not involve multiple types of resins, only the qualitative evaluation indicator I was conducted, and there was no need to conduct the quantitative evaluation indicator I.

The specific test results of the qualitative evaluation indicator I of L₁ resin in this example were as follows:

| | | | Dielectric property |
|---|---|---|---|
| | | Parameter based | parameter at |
| | | on stress-strain | working frequency |
| Rheological property parameter | | test | (50 Hz, 20° C.) |
| shear frequency | complex viscosity Pa · s | tensile strength | relative dielectric |
| (0.01 rad/s, 120° C.) | 1.19 × 10⁵ | 13.46 MPa | constant 2.25 |
| (0.01 rad/s, 150° C.) | 4.20 × 10⁴ | elongation at | dielectric loss angle |
| (0.01 rad/s, 180° C.) | 1.59 × 10⁴ | break 531.7% | tangent 1 × 10⁻⁴ |
| (1 rad/s, 120° C.) | 2.04 × 10⁴ | | |
| (10 rad/s, 120° C.) | 5.65 × 10³ | | |

All criteria of qualitative evaluation indicator I were met. The quantitative evaluation indicator I was skipped to proceed to Step 3.

### Step 3: Optimization of Crosslinking Agent D1 Content

In an internal mixer at a temperature of 110° C. and a speed of 50 r/min, LDPE particles were added and mixed until completely melted. The antioxidant 300 was added to the internal mixer and mixed for 5 minutes. Then a certain amount of DCP was added and mixed for 3 minutes to prepare crosslinkable polyethylene. A certain amount of the crosslinkable polyethylene was hot pressed in a platen vulcanizer at 110° C. for 15 minutes for molding, after which it was transferred to a platen vulcanizer at 175° C. and pressurized to 15 MPa for crosslinking for 30 minutes, and then removed and transferred to a water-cooled platen vulcanizer under the same pressure to cool, resulting in XLPE samples of different specifications. The samples made from the x phr crosslinking agent/0.3 phr antioxidant 300/base resin blends were tested in two steps, where x was a custom variable sequence: x₁=1.6, x₂=1.7, x₃=1.8, and x₄=1.9.

Optimization was conducted through two levels of indicators: qualitative evaluation indicator II and quantitative evaluation indicator II.

The qualitative evaluation indicator II included: two parameters, gel content and thermal elongation, which characterized the degree of crosslinking; and tensile strength and elongation at break parameters obtained based on a stress-strain test. The testing method for the qualitative evaluation indicators was based on the standard JB/T 10437-2004. The requirements were gel content ≥82%, load elongation ≤80%, tensile strength ≥20 MPa, and elongation at break ≥500%.

| | | | Tensile | Elongation |
|---|---|---|---|---|
| | Gel | Thermal | strength | at break |
| Material | content | elongation | (MPa) | (%) |
| x₁ = 1.6 | 77.8% | 90% | 26.3 | 586.2 |
| x₂ = 1.7 | 81.6% | 80% | 25.3 | 607.4 |
| x₃ = 1.8 | 85.0% | 75% | 25.8 | 586.8 |
| x₄ = 1.9 | 85.3% | 70% | 25.9 | 572.7 |

After testing, when x₃=1.8 and x₄=1.9, all of the above indicators were met, and the materials were considered qualified, proceeding to the quantitative evaluation indicator II.

Testing method for the gas production property during crosslinking under quantitative evaluation indicator II: Approximately 5 g of the formulation materials to be tested were weighted using a precision balance, and the weight of the materials was accurately measured before the reaction. After the hot press molding and the crosslinking reaction in the platen vulcanizer, the materials were placed in a vacuum oven at −0.1 MPa and 80° C. and degassed for 72 hours. The weight of the materials was accurately measured again to obtain the gas production amount, the theoretical gas production amount, and the degassing residual amount.

gas production amount

| | gas production | theoretical gas | degassing |
|---|---|---|---|
| | amount at | production | residual |
| material | 72 h(%) | amount(%) | amount(%) |
| x₃ = 1.8 | 1.64 | 1.76 | 92.5% |
| x₄ = 1.9 | 1.72 | 1.86 | 93.2% |

### Step 4: Optimization of Antioxidant Formulation

Two antioxidants could be used alone or in combination. Specifically, the samples made from y phr antioxidant AO₁/z phr antioxidant AO₂/xₙ phr crosslinking agent/base resin blends were tested in two steps, with the sample preparation method consistent with that in Step 3. y was a custom variable sequence: y₁=0.3, y₂=0.15, y₃=0; and z was a custom variable sequence: z₁=0, z₂=0.15, z₃=0.3. Optimization was conducted through two levels of indicators: qualitative evaluation indicator III and quantitative evaluation indicator III.

The qualitative evaluation indicator III included: two parameters, i.e., gel content and thermal elongation, which characterized the degree of crosslinking; and tensile strength and elongation at break parameters before and after aging obtained based on an air aging property test. The testing method was specifically in accordance with the standard JB/T 10437-2004. The requirements were gel content ≥82%, load elongation ≤80%, tensile strength before aging ≥20 MPa, elongation at break before aging ≥500%, and the change rates of tensile strength and elongation at break after aging were less than ±20%.

| | | | | | After aging | |
|---|---|---|---|---|---|---|
| | | | Before aging | | Change | Change |
| | | | Tensile | Elongation | rate of | rate of |
| | Gel | Thermal | strength | at break | tensile | elongation |
| Material | content | elongation | (MPa) | (%) | strength(%) | at break (%) |
| y₁ = 0.3 z₁ = 0 | 87.4% | 54% | 24.4 | 480.6 | No testing | No testing |
| | | | | | required | required |
| y₂ = 0.15 | 88.8% | 63% | 25.3 | 593.9 | 2.42 | −8.72 |
| z₂ = 0.15 | | | | | | |
| y₃ = 0 z₃ = 0.3 | 88.5% | 65% | 24.5 | 542.5 | −0.94 | 1.69 |

Two materials with y₂=0.15 and z₂=0.15, as well as y₃=0 and z₃=0.3, simultaneously met the above indicators and were considered qualified, proceeding to the quantitative evaluation indicator III. The crosslinking reaction kinetic curve was shown in FIG. 2, with the material (y₂=0.15, z₂=0.15) having a higher priority. The material (y₃=0, z₃=0.3) was in secondary priority and served as a candidate to be tested.

### Step 5: Verification of Electrical Properties

The verification of electrical properties was a qualitative evaluation, specifically including: conductivity, dielectric loss factor, relative dielectric constant, and alternating current breakdown strength. The testing methods were in accordance with the standard JB/T 10437-2004, with the following requirements:

| | | |
|---|---|---|
| Electrical properties: | | |
| Relative dielectric constant (50 Hz, 20° C.) | | ≤2.35 |
| Dielectric loss factor (50 Hz, 20° C.) | | ≤5 × 10⁻⁴ |
| Breakdown strength (20° C.) | | |
| (Thickness 100 μm) | MV/m | ≥110 |
| (Thickness 1 mm) | MV/m | ≥30 |
| Volume resistivity (20° C.) | Ω · m | ≥1 × 10¹⁴ |

The material (y₂=0.15, z₂=0.15) met requirements for all of the above parameters, resulting in the final formulation, wherein the base resin is L1, the crosslinking agent is D1 with a content of 1.8 phr, and the antioxidant is AO1 in combination with AO2 each with a content of 0.15 phr.

The beneficial effects of the present disclosure are as follows. Compared to the conventional art, as previously mentioned, the present disclosure provides specific requirements for the key properties of the base resin, provides clear guiding methods, and fully limits the impurity content, the gel point content, and the polar groups of the base resin based on the electrical properties, avoiding the blind selection of the base resin and preventing failures of formulations due to insufficient assessment of key properties, which reduces the waste of subsequent experimental workload and significantly increases the success rate of formulation design.

Based on the inventor's long-term experimental exploration, the optimization of the crosslinking agent formulation cannot overlook the influence of the added antioxidant. In the optimization of the crosslinking agent formulation in the present disclosure, 0.3 phr of antioxidant 300 is used for cooperation. The unique molecular structure of this antioxidant allows it to function as both the primary and secondary antioxidants, demonstrating representativeness and compatibility in functionality. Therefore, the optimization phase of the crosslinking agent is more universal, providing a reliable crosslinking agent formulation optimization structural foundation for subsequent antioxidant formulation optimization, thus reducing potential waste of human resources, material resources, and time.

The three levels of quantitative evaluation indicators in the present application are used to optimize the base resin, the crosslinking agent formulation, and the anti-aging formulation, thereby promoting the formulation design towards high electrical resistance, easy degassing, scorch resistance, and aging resistance according to the weighted priority. This enhances the key properties that are of general concern in the research and development of insulating materials for high-voltage alternating current cables, facilitates the research and development of crosslinked polyethylene insulating materials for alternating current cables at higher voltage levels, and aids in the iterative upgrade of crosslinked polyethylene insulating materials for fixed-voltage grade alternating current cables.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims.

1. A method for designing and optimizing a formulation of an XLPE insulating material for a high-voltage alternating current cable, comprising the following steps:
step 1: determining initial elements of the formulation, including types of a base resin, an antioxidant, and a crosslinking agent;
step 2: based on the base resin selected in the step 1, testing a sample prepared by hot press molding the pure base resin, and conducting optimization sequentially through two levels of indicators: qualitative evaluation indicator I and quantitative evaluation indicator I, to obtain an optimized base resin, wherein the qualitative evaluation indicator I includes a rheological property parameter, a tensile strength parameter, an elongation at break parameter, and dielectric loss angle tangent and relative dielectric constant parameters, and the quantitative evaluation indicator I includes a two-parameter Weibull distribution parameter of alternating current breakdown field strength;
step 3: preparing multiple groups of crosslinking agent/antioxidant/base resin blends, wherein mass fractions of the antioxidant and the base resin in the multiple groups remain the same, while mass fractions of the crosslinking agent in the multiple groups are arranged in an ascending sequence, testing samples of the blends from each group, and conducting optimization through two levels of indicators: qualitative evaluation indicator II and quantitative evaluation indicator II, to obtain an optimized crosslinking agent formulation, wherein the qualitative evaluation indicator II includes a gel content, a thermal elongation, a tensile strength parameter, and an elongation at break parameter, and the quantitative evaluation indicator II includes a crosslinking gas production property parameter;
step 4: preparing multiple groups of crosslinking agent/antioxidant/base resin blends, wherein mass fractions of the crosslinking agent and the base resin in the multiple groups remain the same, while mass fractions of the antioxidant in the multiple groups are arranged in an ascending sequence, testing samples of the blends from each group, and conducting optimization sequentially through two levels of indicators: qualitative evaluation indicator III and quantitative evaluation indicator III, to obtain an optimized antioxidant formulation, wherein the qualitative evaluation indicator III includes the gel content, the thermal elongation, and the tensile strength parameter and the elongation at break parameter before and after aging, and the quantitative evaluation indicator III includes a characteristic parameter of a crosslinking reaction kinetic curve; and
step 5: verifying through electrical properties, if parameter measurement results all meet requirements, the formulation of the material of the material is considered as an optimized formulation, and if not, returning to test a next candidate to be tested.

2. The method of claim 1, wherein in the steps 2, 3, or 4, the qualitative evaluation involves judging whether the parameter measurement results meet the qualitative evaluation indicator I, the qualitative evaluation indicator II, or the qualitative evaluation indicator III, if yes, the material is considered qualified, and if not, the material is abandoned, and a next candidate material is subjected to the qualitative evaluation, until all candidate materials have undergone testing for the qualitative evaluation indicator I, the qualitative evaluation indicator II, or the qualitative evaluation indicator III, and the material meeting the qualitative evaluation indicator is selected to undergo testing for the quantitative evaluation indicator I, the quantitative evaluation indicator II, or the quantitative evaluation indicator III.

3. The method of claim 2, wherein the qualitative evaluation indicator I includes: the rheological property parameter measured with a rotational rheometer, the tensile strength parameter and the elongation at break parameter measured based on a stress-strain test, and the dielectric loss angle tangent and relative dielectric constant parameters measured based on high-voltage Schering bridge.

4. The method of claim 2, wherein the quantitative evaluation indicator I includes the two-parameter Weibull distribution parameter of the alternating current breakdown field strength measured with a cylindrical electrode;
with a characteristic breakdown strength as a first parameter a and a shape parameter as a second parameter b, a priority ranking is conducted on different resins according to values of a; the greater the a, the higher the priority; and with a the same, b is used to further refine the priority ranking, and for materials with the same a, the smaller the b, the higher the priority;
after completing the priority ranking for all resins for optimization, the resin ranked first proceeds to the step 3, while the resins ranked later serves as candidates to be tested.

5. The method of claim 2, wherein in the step 3, samples prepared from x phr crosslinking agent/0.3 phr antioxidant 300/base resin blends are tested and subjected to optimization through two levels of indicators: the qualitative evaluation indicator II and the quantitative evaluation indicator II;
wherein x is a custom variable sequence: x₁, x₂, x₃, x₄ . . . xₙ₁, indicating the mass fraction of the crosslinking agent, x≥0, and n₁ denotes a length of the sequence, i.e., a number of groups of the blends.

6. The method of claim 5, wherein the qualitative evaluation indicator II includes: gel content and thermal elongation parameters characterizing the degree of crosslinking; and the tensile strength parameter and the elongation at break parameter obtained based on a stress-strain test.

7. The method of claim 5, wherein testing the quantitative evaluation indicator II includes: based on the crosslinking gas production property parameter, conducting a priority ranking on materials of all components, wherein the better the gas production property, the smaller the degassing residual amount, the higher the priority, and when the degassing residual amounts are the same, the smaller the value of x, the higher the priority; and after completing the priority ranking for all materials for optimization: x₁, x₂, x₃, x₄ . . . xₙ₁, where n₁ denotes a length of the sequence, the material ranked first proceeds to the step 4, while the materials ranked later serve as candidates to be tested.

8. The method of claim 2, wherein in the step 4, based on mechanism of action and synergistic effects, the antioxidant is used alone, or two types of antioxidants are used in combination.

9. The method for designing and optimizing the formulation of the XLPE insulating material for the high-voltage alternating current cable of claim 8, wherein the qualitative evaluation indicator III includes: gel content and thermal elongation parameters characterizing the degree of crosslinking; and the tensile strength parameter and the elongation at break parameter before and after aging obtained based on an air aging property test.

10. The method of claim 8, wherein testing the quantitative evaluation indicator III includes: based on the characteristic parameter of the crosslinking reaction kinetic curve, conducting a priority ranking on materials of all components, wherein the further to the right the peak point of the crosslinking reaction kinetic curve, the higher the priority, when the crosslinking reaction kinetic curves coincide, the higher the gel content in the qualitative evaluation indicator III, the higher the priority, and when the crosslinking reaction kinetic curves coincide and the gel contents in the qualitative evaluation indicator III are the same, the higher the elongation at break in the qualitative evaluation indicator III, the higher the priority; and after completing the priority ranking for materials of all components to be optimized, the material ranked first proceeds to the step 5.
